(19) United States
(12) Patent Application Publication
(10) Pub. No.: US 2021/0338929 A1
(43) Pub. Date: Nov. 4, 2021

(54) IMPLANTABLE INFUSION PUMP REFILL PORT TACTILE FEEDBACK FEATURE

(71) Applicant: Medtronic, Inc., Minneapolis, MN (US)

(72) Inventors: Nicholas R. Whitehead, Lake Elmo, MN (US); Peter J. Larson, Broomfield, CO (US); Christopher H. Rogers, Plymouth, MN (US); Amanda A. Nowacki, Onalaska, WI (US); Marc A. Crepeau, Maple Grove, MN (US); Joel A. Anderson, Brooklyn Park, MN (US); Forrest C.M. Pape, New Brighton, MN (US); Elizabeth A. Fehrmann, Falcon Heights, MN (US); Brandon J. Johnson, Andover, MN (US); Luis Fesser, Saint Paul, MN (US)

(21) Appl. No.: 16/864,867

(22) Filed: May 1, 2020

(51) Int. Cl. A61M 5/168; A61M 5/142; A61M 5/162

(52) U.S. Cl. CPC A61M 5/16809; A61M 5/14276; A61M 5/162; A61M 2205/582; A61M 2005/14208; A61M 2205/52; A61M 2205/18; A61M 2209/045; A61M 2205/3523; A61M 2205/8206

(57) ABSTRACT

Embodiments of the present disclosure provide a system and method configured to provide feedback to a user during a refill procedure for an implantable medical device via an external refilling apparatus through the use of resilient tactile feedback element within the refill port chamber that provides tactile feedback to a user of a refill needle of a location of the needle within the refill port.

### FIELD

The present disclosure relates generally to implantable medical devices, and more particularly to a system and method configured to provide feedback to a user when refilling of a fluid reservoir of an implantable medical device with medicament.

### BACKGROUND

A variety of medical devices are used for chronic or long-term delivery of therapy to patients suffering from a variety of conditions, such as chronic pain, tremor, Parkinson's disease, cancer, epilepsy, urinary or fecal incontinence, sexual dysfunction, obesity, spasticity, or gastroparesis. For example, pumps or other fluid delivery devices can be used for chronic delivery of therapeutic medicaments, such as drugs or other agents. Typically, such devices provide therapy continuously or periodically according to programmed parameters. The programmed parameters can specify the therapeutic regimen (e.g., the rate, quantity, and timing of medicament delivery to a patient), as well as other functions of the medical device.

Implantable medical infusion pumps have important advantages over other forms of medicament administration. For example, oral administration is often difficult because the systematic dose of the substance needed to achieve the therapeutic dose at the target site may be too large for the patient to tolerate without adverse side effects. Also, some substances simply cannot be absorbed in the stomach adequately for a therapeutic dose to reach the target site. Moreover, substances that are not lipid soluble may not cross the blood-brain barrier adequately if needed in the brain. In addition, infusion of substances from outside the body requires a transcutaneous catheter, which results in other risks such as infection or catheter dislodgment. Further, implantable medical pumps avoid the problem of patient noncompliance of the patient failing to take the prescribed drug or therapy as instructed.

Implantable medical infusion pumps are typically implanted at a location within the body of a patient (typically a subcutaneous region in the lower abdomen) and are configured to deliver a fluid medicament through a catheter. The catheter is generally configured as a flexible tube with a lumen running the length of the catheter to a selected delivery site in the body, such as the intracranial or subarachnoid space.

Various types of implantable medical pumps are in use for dispensing medication within the body. These devices either have reservoirs which are to be filled for dispensation on a time-release basis, such as an implantable drug dispenser, or have ports for insertion of medication that is dispensed through an implantable catheter, commonly known as an access port. In these devices, the reservoir for receiving medication is commonly sealed with a pierceable septum. A hypodermic needle is inserted through the skin and through the access port and the septum into the reservoir. Once within the reservoir, the medication is dispensed from the syringe.

It is critical to the performance of the refilling process that the needle tip is properly positioned at the desired dispensing location. If the needle is outside the device, medication will be improperly dispensed into the body. This could lead to a fatal or otherwise harmful dose of medicament being injected directly into a subcutaneous pocket surrounding the implantable pump. If the needle opening is within the septum, rather than through the septum, excess pressure in the syringe may be required to dispense medication or the dispensing within the reservoir may be entirely prevented and be improperly dispensed into the body. However, access to the refill port with the needle tip cannot be performed with the benefit of direct visualization because the pump is implanted under the skin of the patient.

### SUMMARY

Embodiments of the present disclosure provide a system and method configured to provide confirmation to a user that a needle has entered a refill port during a refill procedure for an implantable medical device through the use of a resilient tactile feedback element within the refill port chamber that provides tactile feedback to a user of the refill needle of a location of the needle within the refill port.

In an embodiment, an implantable medical pump includes a pump housing configured to be percutaneously implanted into a patient and a medicament reservoir contained within the housing configured to contain a medicament to be delivered to the patient. A refill port can be disposed on an exterior surface of the pump housing to provide percutaneous access to a needle to refill the medicament reservoir with the medicament via a refill port cavity in fluid communication with the medicament reservoir. A needle guide can be disposed and movable within the refill port chamber and a resilient member can be disposed distally of the needle guide in the refill port chamber. The needle guide can be configured to move distally under force of the needle to contact the resilient member to actuate the resilient member from a rest position to provide tactile feedback to the user of the location of the needle within the refill port chamber. The needle guide can also function to protect the resilient member from damage that could be caused by direct contact with the needle as well as to center the contact with resilient member at a center of the resilient member.

In an embodiment, a refill port providing percutaneous access to a needle to refill a medicament reservoir of an implantable medical pump can include a refill port cavity in fluid communication with the medicament reservoir and a septum providing a needle access to the refill port cavity. A needle guide can be disposed and movable within the refill port chamber and a resilient member can be disposed distally of the needle guide in the refill port chamber. The needle guide can be configured to move distally under force of a needle to contact the resilient member to actuate the resilient member from a rest position to provide tactile feedback to the user of the location of the needle within the refill port chamber. The needle guide can also function to protect the resilient member from damage that could be caused by direct contact with the needle as well as to center the contact with resilient member at a center of the resilient member.

The above summary is not intended to describe each illustrated embodiment or every implementation of the subject matter hereof. The figures and the detailed description that follow more particularly exemplify various embodiments.

### BRIEF DESCRIPTION OF DRAWINGS

The disclosure can be more completely understood in consideration of the following detailed description of various embodiments of the disclosure, in connection with the accompanying drawings, in which:

FIG. 1 is a schematic view depicting medicament delivery system in accordance with an embodiment of the disclosure.

FIG. 2 is a cross-sectional view of an implantable medical pump in accordance with an embodiment of the disclosure

FIG. 3 is a block diagram depicting an implantable medical pump in accordance with an embodiment of the disclosure.

FIG. 4 is a cross-sectional view of an implantable medical pump in accordance with an embodiment of the disclosure.

FIGS. 5A-5B are cross-sectional views depicting a refill port of an implantable medical pump in accordance with embodiments of the disclosure.

FIGS. 6A-6B are cross-sectional views depicting a refill port of an implantable medical pump in accordance with embodiments of the disclosure.

FIGS. 7A-7B are cross-sectional views depicting a refill port of an implantable medical pump in accordance with embodiments of the disclosure.

FIG. 8 is a cross-sectional view depicting a refill port of an implantable medical pump in accordance with an embodiment of the disclosure.

FIG. 9 is a cross-sectional view depicting a refill port of an implantable medical pump in accordance with an embodiment of the disclosure.

FIG. 10 is a cross-sectional view depicting a refill port of an implantable medical pump in accordance with an embodiment of the disclosure.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

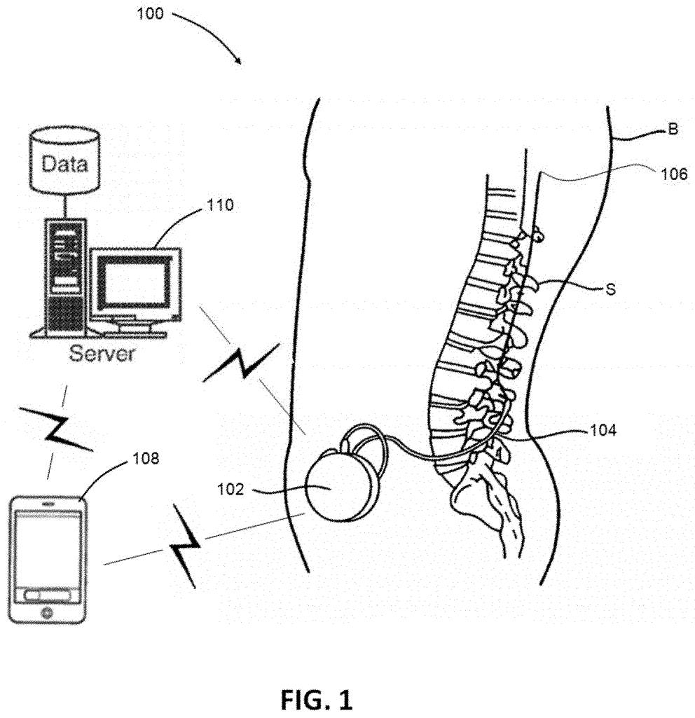

Referring to FIG. 1, a schematic view of a medicament delivery system 100 is depicted in accordance with an embodiment of the disclosure. The medicament delivery system 100 can include an implantable medical pump 102 and a catheter 104. As depicted, the implantable medical pump 102 can be implanted within the body B of a patient. The implantable medical pump 102 can be in fluid communication with the catheter 104 having a distal tip 106 positioned within, for example, the subarachnoid space of the patient's spinal column S to enable intrathecal delivery of medicament through a lumen of the catheter 104. In other embodiments, the distal tip 106 can be positioned within the intracranial space, or other areas within the patient for targeted delivery of medicament. In one embodiment, the medicament delivery system can further include an optional external programmer 108 and/or an optional server 110 configured to communicate with the implantable medical pump 102 and/or with one another. In some examples, pump 102 may provide tactile feedback to the user of the location of a needle as described in this disclosure.

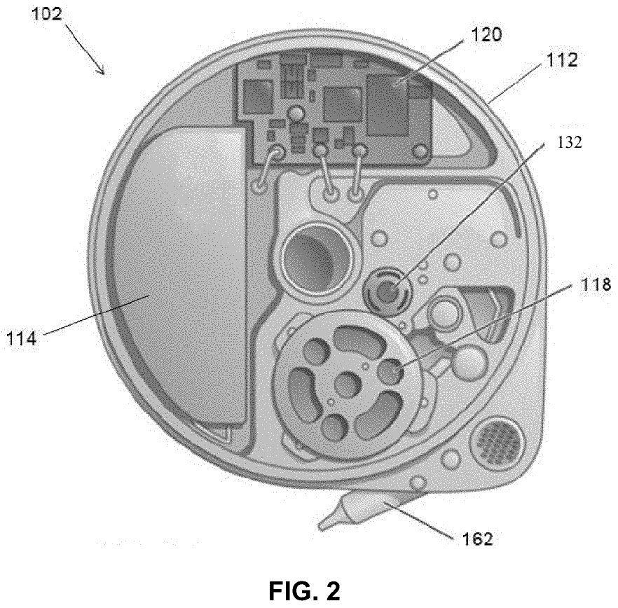

FIG. 2 depicts a cross-sectional plan view of an implantable medical pump 102 in accordance with an embodiment. Implantable medical pump 102 can generally include a housing 112, power source 114, medicament reservoir 116, medicament pump 118, electronic components 120, a refill port 132 and a catheter port 162 for connection to a catheter. The housing 112 can be constructed of a material that is biocompatible and hermetically sealed, such as titanium, tantalum, stainless steel, plastic, ceramic, or the like. In some examples, pump 102 may provide tactile feedback to the user of the location of a needle as described in this disclosure.

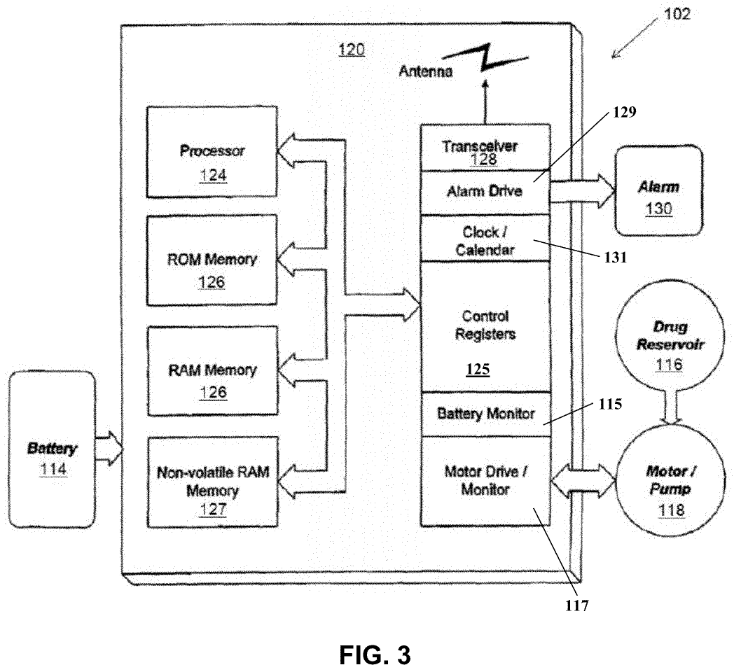

Referring to FIG. 3, a block diagram of an implantable medical pump 102 is depicted in accordance with an embodiment of the disclosure. The electronic components of the device 120 can be carried in the housing 112 and can be in electrical communication with the medicament pump 118 and power source 114. The power source 114 can be a battery, such as a lithium-ion battery. The power source 114 can be carried in the housing 112 and can operate the medicament pump 118 and electronics 120. A battery monitor 115 can monitor a battery power of the battery and a motor drive monitor 117 can monitor operation of the pump motor 118.

The electronic components 120 can include a processor 124, memory 126/127, and transceiver circuitry 128 that can interface with one or more control registers 125. In one embodiment, the processor 124 can be an Application-Specific Integrated Circuit (ASIC) state machine, gate array, controller, microprocessor, CPU, or the like. The electronic components 120 can be generally configured to control infusion of medicament according to programmed parameters or a specified treatment protocol. The programmed parameters or specified treatment protocol can be stored in the memory 126. The transceiver circuitry 128 can be configured to receive information from and transmit information to the external programmer 108 and/or server 110. In one embodiment, the electronic components 120 can be further be configured to operate a number of other features, such as, for example, a patient alarm 130 operable with an internal clock and/or calendar 131 and an alarm drive 129.

The implantable medical pump 102 can be configured to receive programmed parameters and other updates from the external programmer 108, which can communicate with the implantable medical pump 102 through well-known techniques such as wireless telemetry. In some embodiments, the external programmer 108 can be configured for exclusive communication with one or more implantable medical pumps 102. In other embodiments, the external programmer 108 can be any computing platform, such as a mobile phone or tablet. In some embodiments, the implantable medical pump 102 and external programmer 108 can further be in communication with a cloud-based server 110. The server 110 can be configured to receive, store and transmit information, such as program parameters, treatment protocols, drug libraries, and patient information, as well as to receive and store data recorded by the implantable medical pump 102. In some embodiments, pump 102 may provide tactile feedback to the user of the location of a needle as described in this disclosure.

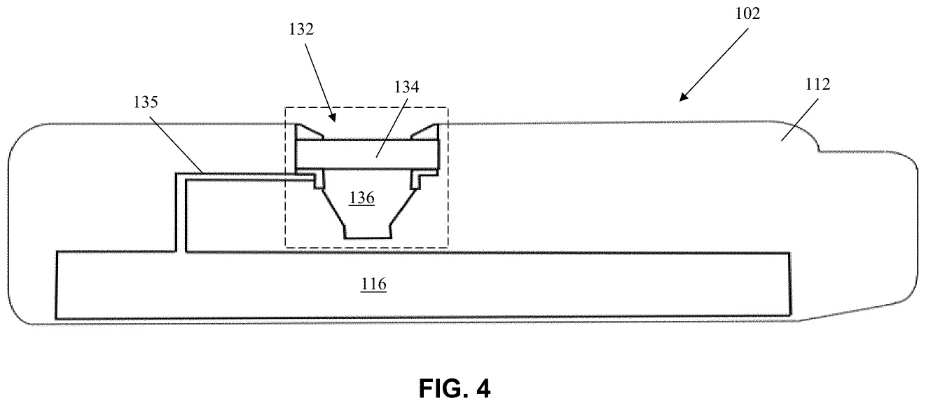

FIG. 4 depicts a cross-sectional view of an implantable medical pump 102 according to an embodiment. A refill port 132 can be disposed on an exterior of the housing 112 with a self-sealing septum 134 enabling a needle to access the refill port cavity 136 percutaneously. A fluid pathway 135 fluidly connects the refill port cavity 136 to the reservoir 116. In some examples, pump 102 may provide tactile feedback to the user of the location of a needle as described in this disclosure.

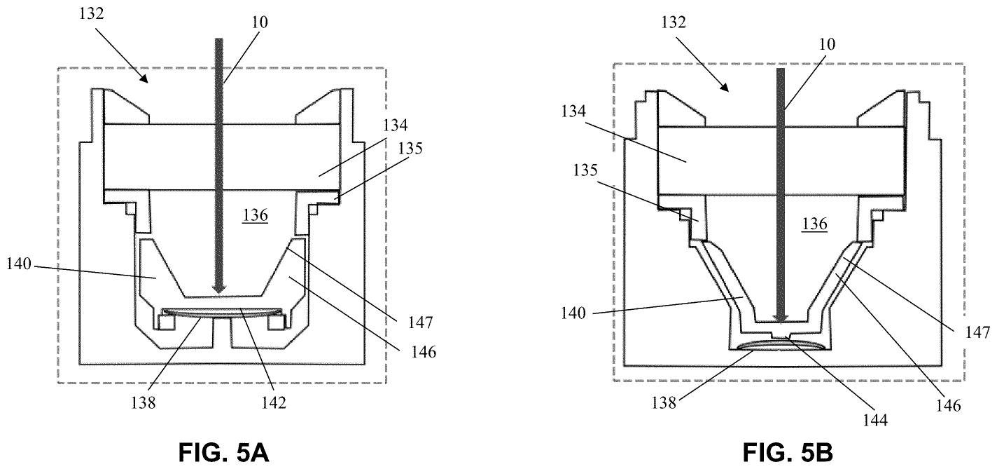

Referring now to FIGS. 5A-5B, cross-sectional views of embodiments of refill ports 132 are depicted with needle 10 inserted through septum 134. A resilient dome 138 can be disposed at a bottom of the refill port cavity 136 and a needle guide 140 disposed in the cavity 136 to contact the resilient dome 138. Prior to any contact with needle guide 140, the resilient dome 138 is in a rest position that the resilient dome is configured to take on when no external forces are acting on the dome. In FIG. 5A the resilient dome 138 is at rest in an inverted configuration and FIG. 5B depicts the resilient dome 138 in another embodiment at rest in a non-inverted or upright position. The resilient dome 138 provides a tactile response to a user inserting the needle 10 by flexing from the rest position and providing a force back towards the needle due to the resilience of the dome 138. In the embodiment of FIG. 5A, the needle guide 140 includes a distal actuation surface 142 that contacts and presses down on the inverted dome 140 causing the dome flex. In the embodiment of FIG. 5B, the needle guide 140 includes an actuation projection 144 that contacts and presses down on the dome 138 causing the dome to flex. In embodiments, resilient dome can be comprised of, for example, tantalum, MP35N®, Elgiloy® or Beta21S.

Needle guide 140 functions to both center the actuation of the resilient dome 138 at the center of the dome and to protect the resilient material of the dome from direct needle 10 contact. Needle guide 140 includes a sloped annular surface 147 that guides a needle 10 that enters the refill port cavity 136 misaligned down to the flat bottom surface of the needle guide. Needle guide 140 can be comprised of a material that can withstand repeated needle contact, such as, for example, a metal material including, e.g., stainless steel, titanium, tantalum, MP35N®, Elgiloy® or Beta21S, among others. Needle guide 140 can translate up and down within the refill port cavity 136 when actuated by the needle 10. In embodiments, needle guide 140 can be generally free floating within refill port cavity 136 but be radially constrained with a body 146 sized to fit within refill port cavity 136 to prevent the needle guide 140 from tilting when moving within the cavity. Needle guide 140 can also be axially restrained from moving up to contact the refill septum 134 by annular rim 135 within refill port cavity 136 that blocks the needle guide 140 from moving axially past the annular rim 135. Needle guide 140 further provides space for fluid to flow between the needle guide and the refill port such that medicament does not get trapped within port.

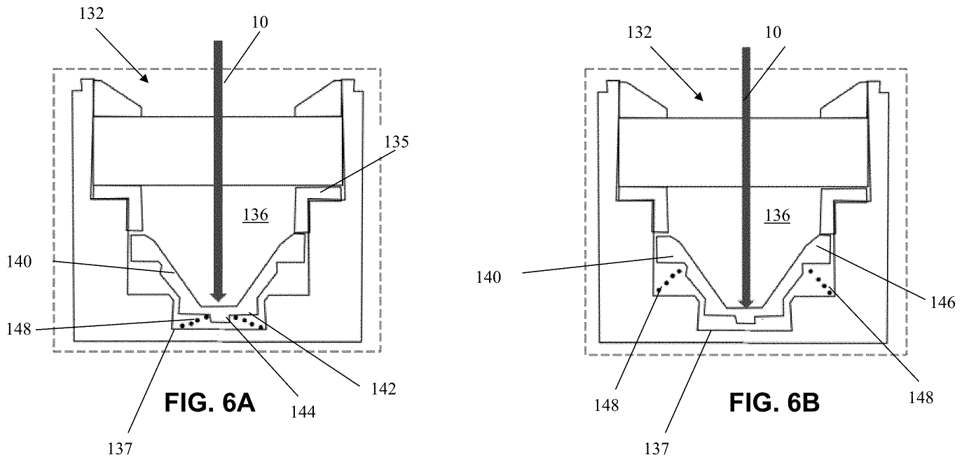

Use of a needle guide 140 such as that described above also enables use of additional resilient members that can provide tactile response to insertion of the needle that, in some embodiments, may not have been useable with a needle alone without such a needle guide 140. For example, FIGS. 6A-6B depict cross-sectional views of refill ports 132 according to another embodiment that employs a spring 148 as a resilient, tactile response element. When the needle guide 140 contacts the spring 148, the spring 148 is compressed from an expanded rest position and provides a tactile compressive force on the needle guide 140 that can be felt by a user operating the needle 10. The user may also feel an additional tactile response if the spring 148 is compressed far enough for the actuation projection 144 of the needle guide 140 to contact a bottom surface 137 of the refill port cavity 136. Although depicted as having the needle guide 140 directly contacting the bottom surface 137 through the spring 148, in other embodiments a resilient dome such as the dome 138 depicted in FIGS. 5A-5B could be disposed at the bottom surface 137 such that the needle guide 140 contacts the dome 138 after compressing the spring 148. In the embodiment of FIG. 6A, the spring 148 is disposed at a bottom of the refill port cavity 136 and is contacted by the actuation projection 144 and/or distal actuation surface 142 of the needle guide 140. In the embodiment of FIG. 6B, the spring 148 is disposed radially around a perimeter of the refill port cavity 136 and is contacted by the body 146 of the needle guide 140. Various spring types that could be employed in embodiments include, for example, a helical spring, a conical spring and a wave spring.

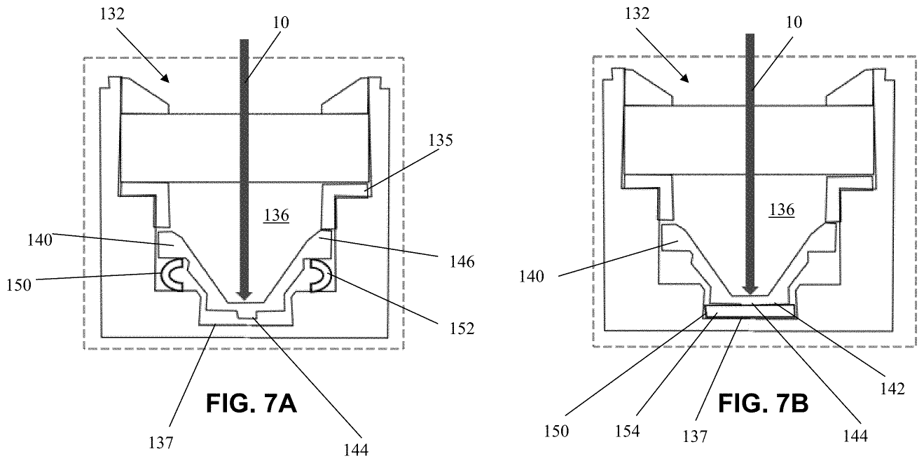

In other embodiments depicted in FIGS. 7A-7B, refill ports 132 can include a compressible solid elastomeric material 150, such as, for example, silicone rubber as a resilient, tactile response element. When the needle guide 140 contacts the compressible material 150 it compresses the material from its natural rest position such that a tactile response is felt by a user operating the needle 10 due to the compressive force between the needle guide 140 and the compressible material 150. In the embodiment of FIG. 7A, the compressible material 150 is provided as a ring 152 having a central opening through which distal surface 142 of the needle guide 140 can extend with the body 146 of the needle guide 140 contacting the ring 152. In the depicted embodiment, the ring 152 has an open, generally “C” shaped cross-section. In other embodiments, the ring 152 can have other cross-sectional configurations, such as a solid, circular cross-section, for example. The user may also feel an additional tactile response if the actuation projection 144 of the needle guide 140 contacts the bottom surface 137 of the refill port cavity 136 through the ring 152. Although depicted as having the needle guide 140 directly contacting the bottom surface 137 through the ring 152, in other embodiments a resilient dome such as the dome 138 depicted in FIGS. 5A-5B could be disposed at the bottom surface 137 to provide additional tactile feedback. In the embodiment of FIG. 7B, the compressible material 150 is configured as a solid disc 154 disposed at the bottom 137 of the refill port cavity 136 that provides tactile feedback when the actuation projection 144 and/or distal surface 142 of the needle guide 140 contact the disc 154.

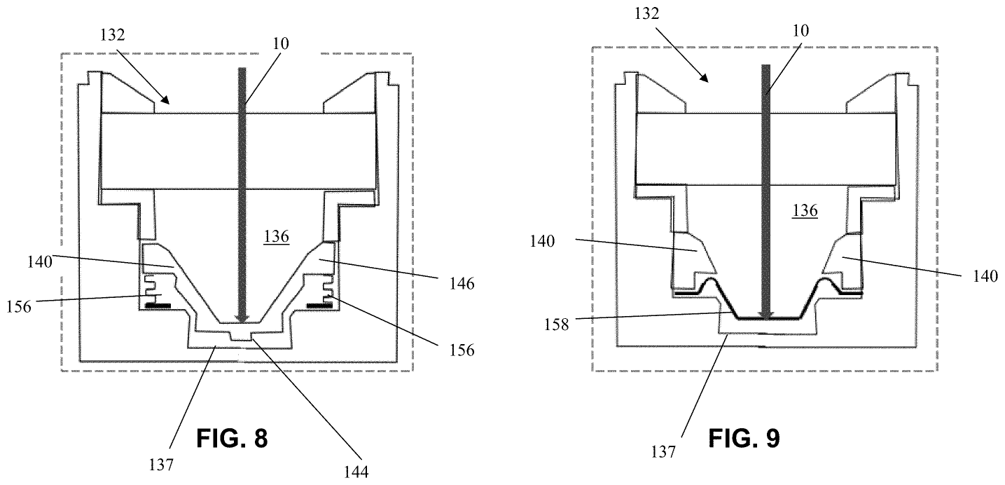

FIG. 8 depicts a refill port 132 according to another embodiment that employs a bellows 156 as a resilient member that provides a tactile response to a user. The tactile response is felt when the body 146 of the needle guide 140 is pressed down by the needle 10 to compress the bellows 156 from the rest position when no external forces are acting on the bellows, which, in turn, provides a force back on the needle guide 140. In the depicted embodiments, bellows 156 can be configured as a ring that extends around the refill port chamber 136 in position to be contacted by the needle guide body 146. The user may also feel an additional tactile response if the actuation projection 144 of the needle guide 140 contacts the bottom surface 137 of the refill port cavity 136 through the bellows 156. Although depicted as having the needle guide 140 directly contacting the bottom surface 137 through the bellows 156, in other embodiments a resilient dome such as the dome 138 depicted in FIGS. 5A-5B could be disposed at the bottom surface 137 such that the needle guide 140 contacts the dome 138 through bellows 156. In other embodiments, a bellows could be provided at the bottom surface 137 of the refill port chamber 136 for direct contact with the actuation projection 144 of the needle guide.

A refill port 132 that utilizes a stamped spring 158 as a resilient tactile feedback element is depicted in FIG. 9. In this embodiment, the needle 10 will directly contact the stamped spring to flex the stamped spring 158 from a rest position down towards the bottom surface 137 of the refill port cavity 137, with the user able to feel the resistance of the stamped spring 158 as it is flexed by the needle. In this embodiment, the needle guide 140 does not move, and serves to guide the needle 10 towards the bottom of the stamped spring 158 if the needle 10 is misaligned upon insertion. Additional tactile feedback can be provided if the stamped spring 158 is flexed downwardly enough to contact bottom surface 137, which could include direct contact with the bottom surface or a resilient dome disposed on bottom surface as discussed above.

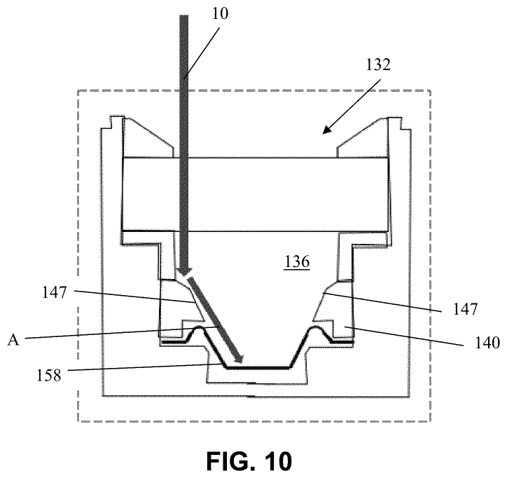

FIG. 10 depicts an example of the alignment aiding aspects of the needle guides disclosed herein. If needle 10 is inserted off center from the refill port cavity 136, it will contact the tapered annular inner surface 147 of needle guide 140 and be deflected towards the center of the needle guide 140 as indicated by the arrow A to guide the needle 110 to the stamped spring 158 below. Although depicted with respect to the needle guide 140 of FIG. 9, such alignment guiding aspects are provided by the angled inner surface of the needle guide 140 of each of the disclosed embodiments. In those embodiments, the angled surface 147 guide the needle towards the bottom of the needle guide 140 to enable the needle to push the needle guide 140 down towards the resilient tactile feedback element of the particular embodiment.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. An implantable medical pump, comprising:
a pump housing configured to be percutaneously implanted into a patient;
a medicament reservoir contained within the pump housing configured to contain a medicament to be delivered to the patient;
a refill port disposed on an exterior surface of the pump housing providing percutaneous access to a needle to refill the medicament reservoir with the medicament via a refill port cavity in fluid communication with the medicament reservoir;
a needle guide disposed and movable within the refill port cavity; and
a resilient member disposed distally of the needle guide in the refill port cavity, wherein the needle guide is configured to move distally under force of the needle to contact the resilient member to actuate the resilient member from a rest position to provide tactile feedback to the user of the location of the needle within the refill port cavity.

2. The implantable medical pump of claim 1, where the resilient member is a resilient dome disposed at a bottom of the refill portion cavity, and wherein the resilient dome is compressible by the needle guide to provide the tactile feedback.

3. The implantable medical pump of claim 1, wherein the resilient member is a spring that is compressible by the needle guide to provide the tactile feedback.

4. The implantable medical pump of claim 3, wherein the spring is disposed at a bottom of the refill port cavity and is compressible by a distal actuation surface of the needle guide.

5. The implantable medical pump of claim 3, wherein the spring is disposed around a perimeter of the refill port cavity and is compressible by an annular body of the needle guide.

6. The implantable medical pump of claim 1, wherein the resilient member comprises a compressible material that is compressed by the needle guide to provide the tactile feedback.

7. The implantable medical pump of claim 6, wherein the compressible material comprises a disc disposed at a bottom of the refill port cavity and is contacted by a distal actuation portion of the needle guide.

8. The implantable medical pump of claim 6, wherein the compressible material comprises a ring disposed around a perimeter of the refill port cavity and is contacted by an annular body of the needle guide.

9. The implantable medical pump of claim 1, wherein the resilient member comprises a bellows.

10. The implantable medical pump of claim 1, wherein the needle guide comprises an annular body and a bottom surface, and wherein the annular body is tapered towards the bottom surface to facilitate guiding the needle towards the bottom surface.

11. A refill port providing percutaneous access to a needle to refill a medicament reservoir of an implantable medical pump, comprising:
a refill port cavity in fluid communication with a medicament reservoir;
a septum providing a needle access to the refill port cavity;
a needle guide disposed and movable within the refill port cavity; and
a resilient member disposed distally of the needle guide in the refill port cavity, wherein the needle guide is configured to move distally under force of a needle to contact the resilient member to actuate the resilient member from a rest position to provide tactile feedback to the user of the location of the needle within the refill port cavity.

12. The refill port of claim 11, where the resilient member is a resilient dome disposed at a bottom of the refill portion cavity, and wherein the resilient dome is compressible by the needle guide to provide the tactile feedback.

13. The refill port of claim 11, wherein the resilient member is a spring that is compressible by the needle guide to provide the tactile feedback.

14. The refill port of claim 13, wherein the spring is disposed at a bottom of the refill port cavity and is compressible by a distal actuation surface of the needle guide.

15. The refill port of claim 13, wherein the spring is disposed around a perimeter of the refill port cavity and is compressible by an annular body of the needle guide.

16. The refill port of claim 11, wherein the resilient member comprises a compressible material that is compressed by the needle guide to provide the tactile feedback.

17. The refill port of claim 16, wherein the compressible material comprises a disc disposed at a bottom of the refill port cavity and is contacted by a distal actuation portion of the needle guide.

18. The refill port of claim 16, wherein the compressible material comprises a ring disposed around a perimeter of the refill port cavity and is contacted by an annular body of the needle guide.

19. The refill port of claim 11, wherein the resilient member comprises a bellows.

20. The refill port of claim 11, wherein the needle guide comprises an annular body and a bottom surface, and wherein the annular body is tapered towards the bottom surface to facilitate guiding the needle towards the bottom surface.
